(19) United States
(12) Patent Application Publication
(10) Pub. No.: US 2026/0240684 A1
(43) Pub. Date: Aug. 20, 2026

(54) CONCENTRATION AID DEVICE

(71) Applicant: Bulledac, Sanary-Sur-Mer (FR)

(72) Inventor: Sophie LEBON, Sanary-Sur-Mer (FR)

(21) Appl. No.: 19/162,096

(22) PCT Filed: Mar. 19, 2024
(86) PCT No.: PCT/FR2024/050322

§ 371 Date: Sep. 4, 2025

(30) Foreign Application Priority Data
Mar. 20, 2023 (FR) FR23/02555

(51) Int. Cl. A61F 9/04

(52) U.S. Cl. CPC A61F 9/045; A41D 20/00; A61F 9/027; A61F 9/029; A63B 71/10; G09B 19/00

(57) ABSTRACT

The present invention relates to a concentration aid device comprising a headband capable of surrounding the forehead and the head of a person, the concentration aid device comprising: a median recess intended to be arranged in front of the eyes of the person, two lobes located on either side of the median recess intended to be placed against, and to cover, the ears of the person, thereby forming two blinders capable of laterally limiting the field of vision of this person, a support strip which is positioned on an inner face of the headband above at least the median recess enabling the headband to be spaced apart from the forehead of the person such that the support strip, together with the median recess, ensures a clear forward field of vision.

### TECHNICAL FIELD

The present invention relates to a device for aiding concentration and isolation for any person of any age (child, adolescent, or adult) who either has difficulty concentrating or simply wishes to isolate themselves, particularly in an enclosed space and when noise or agitation is present around the person, in order to perform a personal activity.

In particular, simply telling a person to “concentrate” often does not make sense to them, especially in the case of children. It is therefore necessary to provide tools that assist the person in concentrating, in particular during the learning phase of the activity for which concentration is required.

### BACKGROUND OF THE INVENTION

To date, there are four types of devices for assisting a person in concentrating on an activity:
noise-cancelling, headset-type devices, as taught in patent application KR20150028792A of 2015, which improve concentration, on the one hand by preventing disturbance to neighboring people, since sound generated inside the device is not transferred to the outside, and, on the other hand, by blocking external noise to provide an environment suitable for concentrating without disturbance by external noise;
glasses or headbands as taught in patent application JPH0990285A describing glasses provided with side obstacles for narrowing the right and left vision angles, or application JPH0990865A describing a headband also equipped with semi-circular side obstacles in the shape of earphones which narrow the left and right vision angles to focus a student's attention forward and also prevent the student from cheating during an exam;
concentration aids based on specific and complex games or equipment, designed to attract and capture the attention of individuals or children, as described in utility certificate applications CN215895737 or CN208954409;
removable concentration screens made up of 3 panels (they can therefore be foldable) that are placed on a table or desk to temporarily create a quiet U-shaped workspace, minimizing auditory and visual distractions to facilitate concentration of the person, such as a student, who places their head in the space thus created between the two side panels forming the branches of the U.

All these known devices certainly allow, each in their own way, a person to concentrate on a personal activity; however, they are either complex and therefore expensive, or, although they may help with concentration, the person is restricted to the activity provided by the device itself, or they fail to satisfy the two criteria necessary for effective concentration, namely silence and visual isolation, so as not to be distracted by surrounding activity.

The state of the art can also be illustrated by the teachings of document WO2011/137463, which discloses a resting headband for a traveler that completely covers the eyes in order to block light from the traveler's eyes, and includes means for attenuating sound, the aim being to isolate the senses of sight and hearing to facilitate rest and falling asleep. Consequently, the headband is unsuitable for performing other activities.

### SUMMARY OF THE INVENTION

The problem therefore is to provide a simple, and consequently inexpensive, device that helps anyone to concentrate on a personal activity, particularly during the learning or performance of a specific task that is independent of the device itself, such as reading or writing for a child, and which requires isolation from both auditory and visual stimuli; furthermore, the device also aims to address the difficulty of making surrounding people understand that the person requires calm and silence to concentrate.

A solution to this problem is a concentration aid device for assisting the concentration of any person, wherein the concentration aid device comprises a headband capable of surrounding the forehead and head of said person, the headband having an inner face intended to be positioned on the forehead and temples of the person and an opposite outer face, and comprising:
a median recess intended to be placed in front of the person's eyes;
two lobes surrounding said recess on either side, intended to be placed against, and to cover, the person's ears;
   and wherein the concentration aid device comprises a support strip positioned on the inner face of the headband above at least the median recess, configured to space the headband away from the forehead of the person, so that said support strip, together with the median recess, enables the forward field of vision to be cleared, said field of vision being restricted laterally by the two lobes forming two blinders capable of laterally limiting the field of vision.

Thus, once the device is worn by the person, the median recess provided in the headband is located in front of the eyes (i.e. opposite the eyes), and at the same time this headband is spaced away from the forehead by the support strip, thus jointly clearing the field of vision. In fact, the support strip creates a space between the headband and the eyes, and the median recess opens the field of vision in this space. It is apparent that the median recess clears the field of vision in the headband, while restricting it to the limits defined by the shape and dimensions of this median recess.

It is also apparent that the median recess defines a cut-out portion (therefore empty of material) in the headband, and when it is stated that the median recess is arranged in front of the eyes, this means that it is the cut-out portion (and therefore the empty space) which is positioned in front of the eyes. In other words, considering that the cut-out portion is delimited by an upper edge, then this upper edge is positioned above the line of sight, the person's eyes being aligned with this line of sight. This median recess is also delimited by side edges (on the right and left) which extend below this line of sight and form the edges of the lobes on either side of the median recess,

Furthermore, the lobes serve to limit this field of vision, and more generally, it is the edges (or ridges) of the median recess that limit this field of vision laterally (to the right and to the left) and upward. Thus, this clearing of the field of vision, within the limit given by the median recess, enables the person to perform an activity (such as reading, writing, drawing, etc.) while being isolated in a kind of visual tunnel.

The present invention further addresses the entire problem posed by allowing any person wishing to concentrate on a personal activity to have a simple, inexpensive device which helps them, in particular when learning or applying a specific task independent of the device itself, to isolate themselves from their environment both acoustically, owing to the lobes and optionally the pads covering the ears, and visually, owing to the side lobes forming blinders which limit the field of vision by only allowing said person to look ahead.

The present invention thus proposes that a person wishing to concentrate on a personal activity “place themselves in a bubble” during this activity, which requires concentration, by putting on their head a device according to the invention.

The present invention also stimulates the learning of concentration because the use of such a device creates an automatic reflex so that the person can concentrate even without this device after a learning phase with it: in the absence of this tool, the person will in fact have the reflex to put their hands to their temples to reduce their field of vision.

In a preferred embodiment, the headband according to the invention comprises:
on the one hand, a piece made from a flexible sheet of elongated shape, narrower in width in the middle, forming the recess, and wider on either side of it, forming the lobes capable of covering the ears of the person,
on the other hand, a fastening system securing the two ends of the part and capable of holding it around the head of the person.

This type of design is particularly advantageous, as it is based on a thin, flexible sheet that can be easily and cheaply manufactured.

According to another feature of the invention, the headband includes on its outer face at least one term expressing silence, such as the word “Shh”, in order to make others understand that the person wearing the device needs isolation: other people thus know that this person wants to concentrate and that they must respect the silence, which addresses one of the objectives of the invention.

According to one feature, the flexible sheet is made of a plastic material.

According to another feature, the fastening system joining the two ends of the piece is of adjustable length so as to allow adaptation to the head of the person.

In a particular embodiment, the concentration aid device includes flexible pads at the lobes, positioned on the inner face of the headband.

In other words, the lobes may have on their inner face flexible pads, such as foam pads, which may, for example, be added or removed using a two-part Velcro type hook-and-loop self-gripping attachment, in order to optimally block the ears of the person wearing the device and thereby reduce their perception of external noise.

According to one possibility, the concentration aid device includes a notch capable of receiving a label for personalizing the device.

In a particular embodiment, the support strip has a thickness of between 1 and 4 centimeters, for example between 1.5 and 2.5 centimeters.

Such a thickness allows the headband to be spaced sufficiently away from the forehead to clear the field of vision, while still maintaining this isolating effect for the person.

According to one possibility, the support strip is made at least partially of foam. Thus, the foam support strip, which comes into contact with the forehead, is sufficiently supple to enhance comfort.

Alternatively, the headband has a lower edge in which the median recess is formed, and an upper edge opposite the lower edge along which the support strip is arranged.

Alternatively, the median recess is delimited by an upper edge and two side edges adjacent to the two respective lobes.

In an advantageous embodiment, the upper edge has a length of between 8 and 12 centimeters, such length being sufficient to cover the spacing between the eyes.

This length of the upper edge is measured along a longitudinal axis parallel to a line of sight, said line of sight being defined as the axis passing through the centers of the two eyes of the person. The upper edge can be straight or curved.

According to one feature, the two side edges each have a length of between 5 and 10 centimeters.

This length of the side edges allows the lobes to extend sufficiently far below the line of sight so as to create the desired tunnel effect.

According to one feature, the median recess is delimited by a bent or curved edge line generally in the shape of an inverted “U”.

According to another feature, the lobes each have a width of between 10 and 20 centimeters, for example between 13 and 18 centimeters.

This width, measured in a vertical orientation on the person, promotes isolation by extending down to the cheeks, below the cheekbones, on either side of the median recess.

The invention also relates to a use of a concentration aid device according to the invention, for aiding the concentration of a person, in which the headband surrounds the forehead and the head of this person, by positioning the median recess in front of the eyes of the person and by positioning the support strip against the forehead so as to space the headband away from the person's forehead and to clear a forward field of vision, said field of vision being laterally restricted by the two lobes.

In an advantageous use, the median recess is delimited by an upper edge, the eyes of the person are aligned along a line of sight, and said headband is positioned on the head of the person so that the upper edge is positioned above the line of sight.

Advantageously, the upper edge is positioned at a given distance above the line of sight, said distance being at most 3 centimeters, and for example between 1 and 2 centimeters.

Such a distance allows a restricted field of vision to be maintained, thus providing the desired tunnel effect. In fact, when the person lowers their head to concentrate on a tabletop activity (for example reading, writing, coloring, drawing, etc.), this upper edge blocks vision in the upper part of the field thereby promoting the desired tunnel effect.

### BRIEF DESCRIPTION OF THE DRAWINGS

Other features and advantages of the invention will become apparent upon reading the detailed description which follows, with reference to the appended figures, said figures being provided by way of non-limiting example.

For the purposes of describing and understanding the invention, and by way of convention and without limiting reference to other reference points such as terrestrial gravity, the vertical, longitudinal and transverse orientations according to the reference points V, L, T shown in the figures, wherein the longitudinal axis Land vertical axis V extend in the plane of FIGS. 1 and 2, are adopted on a non-limiting basis:
the longitudinal orientation L corresponds to the alignment of the eyes of the person wishing to concentrate, and also defines lateral positioning along this longitudinal orientation, as represented in FIG. 5, this longitudinal orientation being considered horizontal when the person stands upright with the head aligned along a vertical axis,
the vertical orientation V is perpendicular to the longitudinal orientation in the plane of FIGS. 1 and 2, and defines positions such as upper, lower, above, and below.
the transverse orientation T, which also defines internal positioning, corresponds to the side facing the person who wishes to concentrate, and the external positioning corresponds to the opposite side, facing away from the person, and then defining the front positioning as the side directed toward the person, as shown in FIG. 4, and toward the device according to the invention as shown in FIG. 3, while the back positioning is opposite the front positioning.

In this manner;

FIG. 1 is a schematic front view of an exemplary embodiment of the invention in an open, flat position, before it is positioned around the head of a person who wishes to concentrate or is instructed to concentrate;

FIG. 2 is a schematic rear view of the exemplary embodiment of a device according to the invention shown in FIG. 1;

FIG. 3 is a schematic perspective front view of the same embodiment of a device according to the invention as in FIGS. 1 and 2, but in the closed position, as it appears when placed around the head of a person who wishes to concentrate or is instructed to concentrate, as shown in FIGS. 4 and 5;

FIG. 4 is a schematic front perspective view of the same embodiment of a device according to the invention as shown in FIGS. 1 to 3, in the closed position as in FIG. 3, and positioned around the head of a person who wishes to concentrate or is instructed to concentrate;

FIG. 5 is a schematic side perspective view of the same embodiment of a device according to the invention as shown in FIGS. 1 to 4 in the closed position and positioned as shown in FIG. 4 around the head of a person who wishes to concentrate or is instructed to concentrate;

FIG. 6 is a view of the device corresponding to FIG. 1, without inscriptions or pictograms;

FIG. 7 is a view of the device corresponding to FIG. 3, without inscriptions or pictograms;

FIG. 8 is a partial view of the device corresponding to FIG. 2, including a cross-sectional view along axis A-A;

FIG. 9 is a view corresponding to FIG. 5, without inscriptions or pictograms, showing a person with the left eye, left ear, and forehead visible through transparency, as well as the support strip used to space the device away from the forehead;

FIG. 10 is a view of the device corresponding to FIG. 4, without inscriptions or pictograms, showing a person with both eyes, pads, and the support strip visible through transparency.

### DETAILED DESCRIPTION OF AN EXEMPLARY EMBODIMENT OF THE INVENTION

In this description, identical, similar or analogous elements are designated by the same reference numerals.

The concentration aid device 100 for assisting the concentration of any person 12 comprises, regardless of the embodiment of the invention, a headband 1 capable of surrounding the forehead and head 13 of this person 12 and comprising:
a median recess 3 intended to be positioned in front of the eyes of the person 12 and to clear their forward field of vision 10,
two lobes 4₁, 4₂ surrounding said recess 3 on either side, intended to be placed against and to cover the ears of the person 12, if not to block them, and forming two blinders capable of laterally limiting the field of vision 10 of the person 12 to an angle of at most 80 degrees centered on either side of the median plane of the person 12 and therefore of the device.

The median recess 3 is delimited by an upper edge 30 extending substantially parallel to the longitudinal orientation L, and by two side edges 31, 32 disposed respectively to the right and left of the median plane of the person 12. The side edges 31, 32 form edges of the lobes 4₁, 4₂. The upper edge 30 together with two side edges 31, 32 defines a bent or curved edge line which is generally in the shape of an inverted “U”.

The upper edge 30 may be straight or curved, and it extends between two bends 33, 34 forming curvatures connecting the upper edge 30 and the respective side edges 31, 32. The side edges 31, 32 extend in directions inclined relative to the longitudinal orientation Lat angles of approximately 90 to 135 degrees.

As shown in FIG. 7, the upper edge 30 has a length L30, measured along the longitudinal orientation L (between the two bends 33, 34), which can be between 8 and 12 centimeters. The side edges 31, 32 each have a length L31, L32, which is the same for both side edges 31, 32, and can be between 5 and 10 centimeters.

In the description of the exemplary embodiment which follows, the headband 1 according to the invention comprises:
on the one hand, a part 11 made from a flexible sheet, composed of a plastic-type material, of elongated shape in the longitudinal direction L defined above, of width in the direction V, narrower l₃ in its middle portion, forming the recess, and wider l₄ on either side, forming the lobes 4₁, 4₂ capable of covering the ears of the person and forming two blinders,
on the other hand, a fastening system 2 securing the two ends of the part 11 and capable of holding it around the head of the person 12.

In alternative embodiments not described here, the headband 1 can, for example, be preformed according to different sizes to correspond to given head sizes, similar to hats and helmets: the device 100 is then positioned on the head of the person 12 by slipping it on and placing it over the head, like a hat or helmet.

The headband 1 has an inner face 15 intended to be positioned on the forehead and temples of the person 12, and an outer face 16 opposite the inner face 15 and intended to be visible from the outside. For the remainder of the description, the outer face 16 of the headband 1 can be likened to the outer face of the part 11 and to the outer face of the device 10, and likewise, the inner face 15 of the headband 1 can be likened to the inner face of the part 11 and to the inner face of the device 10.

The lobes 4₁, 4₂ each have a width 14 (corresponding to the greatest width of the part 11 as described above), where this width 14 can be between 10 and 20 centimeters, and for example between 13 and 18 centimeters. The headband has a width l₃ above the middle recess 3 (corresponding to the narrowest width of the part 11 as described above), which can be between 2 and 8 centimeters.

The headband 1 has a lower edge 17 in which the median recess 3 is formed, the lower inner edge 17 being turned downward, towards the nose of the person 12, and it has an upper edge 18 opposite the lower edge 17 and turned upward, towards the forehead of the person 12. For the remainder of the description, the lower edge 17 of the headband 1 can be likened to the lower edge of the part 11, and the upper edge 118 of the headband 1 can be compared to the upper edge 18 of the part 11.

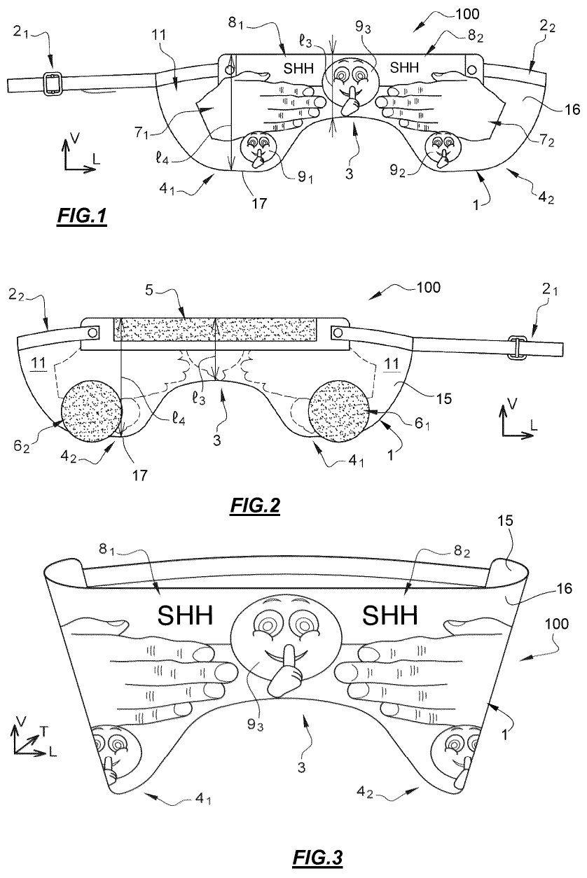

FIG. 1 schematically represents a view of the outer face 16 of an exemplary embodiment of a device 100 according to the invention in which the headband 1 is formed from a part 11 made from a flexible sheet of elongated shape in the open flat position, and includes on its outer face 16 at least the inscription of one or more terms 8₁, 8₂ expressing silence, such as the word “Shh” shown in the Figures as an example, and even repeated twice: in this case, these are arranged on either side of at least the inscription of a pictogram 9 also expressing silence, such as a “smiley” 9₃ as shown in the Figures, and positioned above the median recess 3.

As shown in FIG. 1, the two lobes 4₁, 4₂ may each include, on their outer face 16, the inscription of a graphic 7₁, 7₂ expressing concentration, or indicating at least partial visual obstruction, such as a representation of an open hand with the back of it facing forward, shown by way of example, and:
either these graphics 7₁, 7₂ are necessarily opaque and are even associated with opaque covers located below if, on the one hand, the flexible sheet forming the part 11 is transparent, and, on the other hand, the height of these graphics 7₁, 7₂ is insufficient for the lobes 4₁, 4₂ to act effectively as blinders,
or, when part 11 is itself opaque, these graphics 7₁, 7₂ can be stylized only and do not require additional opaque covers as shown elsewhere in the Figures.

As shown, other pictograms 9₁, 9₂, such as smileys, of the same type as that positioned above the median recess 3 but preferably of smaller dimensions, can also be placed on each lobe 4₁, 4₂ close to their lower edge 17 and below the graphics 7₁, 7₂ expressing concentration, such as hands.

Said inscriptions can be printed directly on the part 11 or on stickers or sheets which are subsequently adhered or laminated onto said part 11.

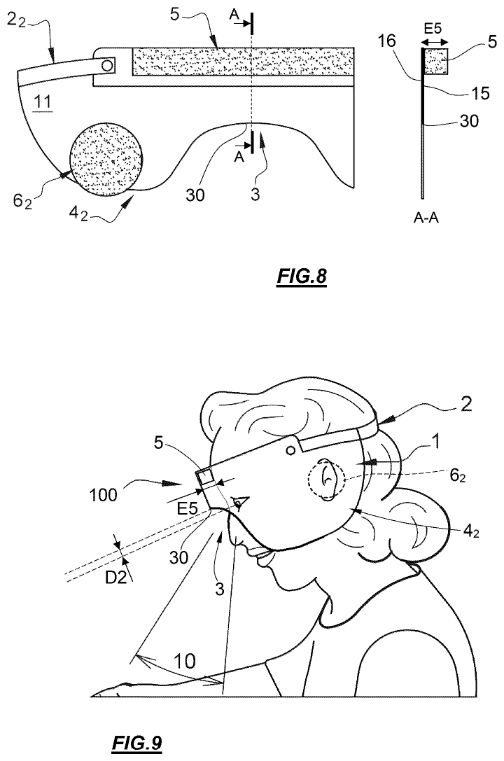

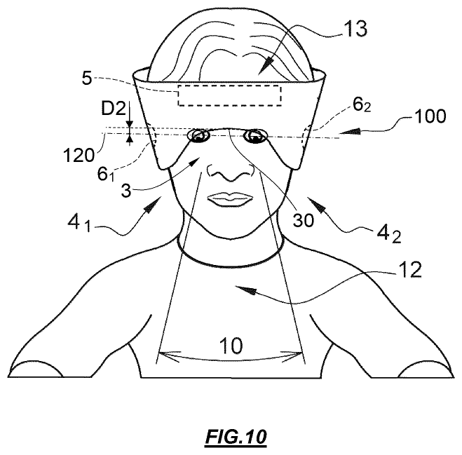

FIG. 2 shows a schematic rear view (i.e., from the side of the inner face 15) of the same exemplary embodiment of the device 100 shown in FIG. 1: this inner face 15 of the headband 1 is intended to be positioned on the forehead and temples of the person 12. As can be seen in this FIG. 2, the device 100 includes a support strip 5 which is positioned on this inner face 15 of the headband 1 or of the part 11, and which is located towards the upper edge 18 of the headband 1, at least above the recess 3. With reference to FIGS. 8 and 9, this support strip 5 has a thickness E5, for example between 1 and 4 centimeters, and in particular between 1.5 and 2.5 centimeters.

Thus, this support strip 5 allows the headband 1 to be spaced away from the forehead of the person 12 by a distance corresponding to the thickness E5. In this way, the support strip 5 prevents the headband 1 from being pressed against the forehead and therefore being too close to the eyes, and together with the median recess 3, allows the forward field of vision 10 to be cleared.

This support strip 5 is preferably made of foam so as to be compressible, and provides, on the one hand, greater comfort of contact between the device 100 and the forehead of the person 12 and, on the other hand, spaces the headband 1 away from the forehead. This support strip 5 extends along the upper edge 18 of the headband 1, over a given length which can be between 5 and 15 centimeters.

On the other hand, the device 100 may include, in a preferred embodiment, flexible pads 6 on the lobes 4₁, 4₂; these pads 6 can be glued or they can be added or removably attached, for example, by a two-part Velcro type hook-and-loop self-gripping system, so as to optimally block the ears of the person wearing the device.

These pads 6 can be made of foam and they are intended to be positioned against the ears of the person 12; they serve to reduce the perception of ambient noise around the person 12. In one option, these pads 6 are mounted on bases that secure the foam pads to the ears. These bases can be made of a plastic material, for example, molded plastic.

The headband 1 comprises a fastening system 2 of any known type, connecting the two ends of the elongated part 11, adjustable in length. As shown in FIGS. 1 and 2, this fastening system 2 can consist of two parts 2₁, 2₂, each secured to the upper part of one end of the part 11 and capable of being connected together once the device has been positioned around the head 13 of the person 12, and behind their head, with the length of the fastening system 2 adjustable to the head size of the person and the desired tightness.

FIG. 3 is a front perspective view of the exemplary embodiment of a device 100 shown in FIGS. 1 to 2, in the closed position.

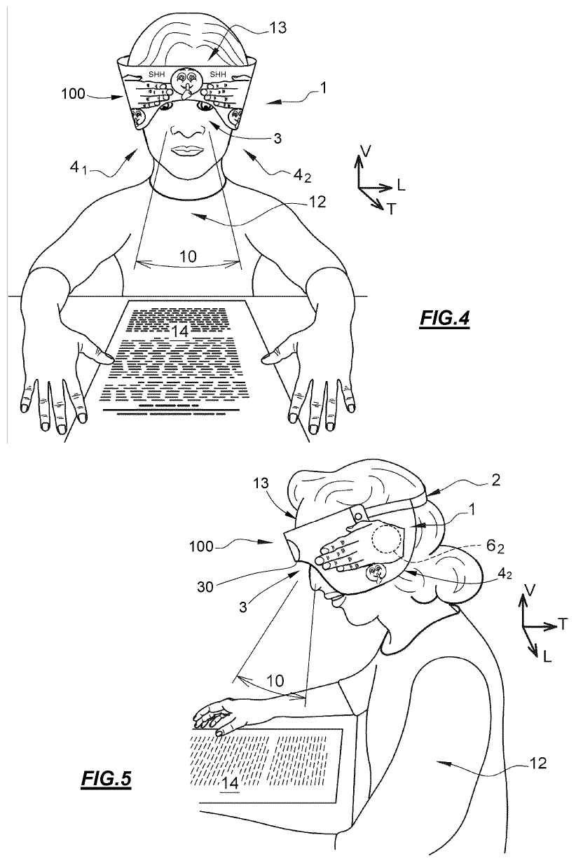

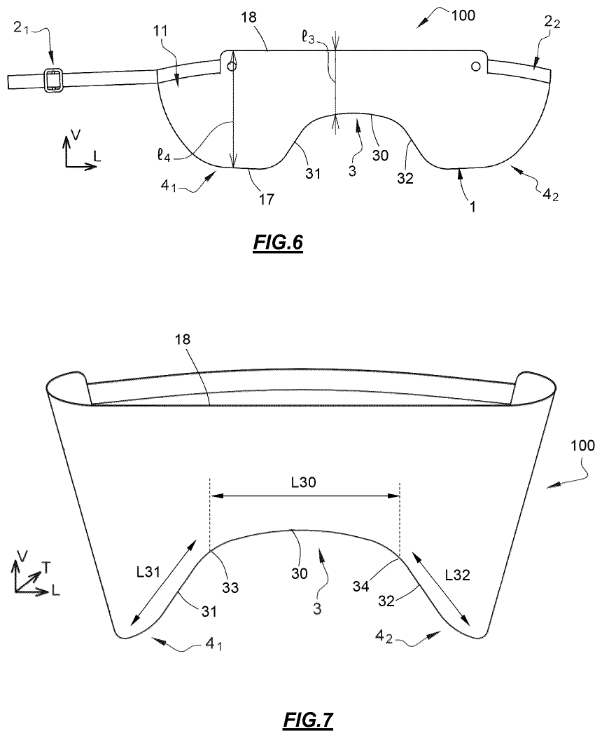

In FIGS. 4 and 10, a device 100 is shown in the closed position, from the front, positioned around the head of a person 12 who wishes to concentrate or is instructed to concentrate on a tabletop activity, for example reading a text 14 placed in front of them. FIG. 10 illustrates that, in use, the headband 1 is positioned on the head 13 of the person 12 so that the upper edge 30 is located above a line of sight 120, the eyes of the person 12 being aligned with this line of sight 120, which is parallel to the longitudinal orientation L. More specifically, the upper edge 30 is positioned at a given distance D2 above the line of sight 120, this distance D2 being at most 3 centimeters, and for example, between 1 and 2 centimeters. This distance D2 is also shown in FIG. 9.

In FIGS. 5 and 9, a device 100 is illustrated in the closed position and positioned as in FIGS. 4 and 10 around the head of the person 12 in side view to show what has been previously described regarding the positioning of the lobes 4₁, 4₂ on the ears of the person 12, which also constitute blinders capable of laterally limiting the field of vision 10 to an opening just sufficient to read the text 14, and also regarding the spacing of the headband 1 from the forehead achieved by means of the support strip 5 of thickness E5.

Finally, the device 100 may include a notch, not shown in the figures, capable of receiving a label for personalizing this device, such as the first name of the person 12 who uses it.

What is claimed is:

1. A concentration aid device (100) for assisting the concentration of any person (12), wherein said concentration aid device (100) comprises a headband (1) capable of surrounding the forehead and head (13) of the person (12), said headband (1) having an inner face (15) intended to be positioned on the forehead and temples of the person (12) and an opposite outer face (16), and comprising:
a median recess (3) intended to be placed in front of the eyes of the person (12);
two lobes (41, 42) surrounding said recess (3) on either side, intended to be placed against, and to cover, the ears of the person (12);
and wherein said concentration aid device (100) comprises a support strip (5) which is positioned on the inner face (15) of the headband (1) above at least the median recess (3) and configured to space the headband (1) away from the forehead of the person (12), so that said support strip (5) makes it possible, together with the median recess (3), to clear a forward field of vision (10), which field of vision (10) is restricted laterally by the two lobes (41, 42) which form two blinders capable of laterally limiting the field of vision (10).

2. The concentration aid device (100) according to claim 1, wherein the headband (1) includes:
on the one hand, a part made from a flexible sheet (11) of elongated shape, having a width (l3) narrower in the middle, forming the recess (3), and a width (l4) greater on either side, forming the lobes (41, 42),
on the other hand, a fastening system (2) securing the two ends of the part made from the flexible sheet (11) when positioned around the head (13) of the person (12).

3. The concentration aid device (100) according to claim 2, wherein the flexible sheet (11) is made of plastic.

4. The concentration aid device (100) according to claim 2, wherein the fastening system (2) securing the two ends of the part made from the flexible sheet (11) is adjustable in length.

5. The concentration aid device (100) according to claim 1, wherein it the concentration aid device (100) includes one or more flexible pads (6) on the lobes positioned on the inner face (15) of the headband (1).

6. The concentration aid device (100) according to claim 1, wherein the concentration aid device (100) includes a notch capable of receiving a label for personalizing the device.

7. The concentration aid device (100) according to claim 1, wherein the support strip (5) has a thickness(ES) of between 1 and 4 centimeters.

8. The concentration aid device (100) according to claim 1, wherein the support strip (5) is made at least partially of foam.

9. The concentration aid device (100) according to claim 1, wherein the headband (1) has a lower edge (17) in which the median recess (3) is formed, and an upper edge (18) opposite the lower edge (17) along which the support strip (5) is arranged.

10. The concentration aid device (100) according to claim 1, wherein the median recess (3) is delimited by an upper edge (30) and two side edges (31) adjacent to the two lobes (41, 42) respectively.

11. The concentration aid device (100) according to claim 10, wherein the upper edge (30) has a length (L30) of between 8 and 12 centimeters.

12. The concentration aid device (100) according to claim 10, wherein the two side edges (31, 32) each have a length (L31, L32) of between 5 and 10 centimeters.

13. The concentration aid device (100) according to claim 1, wherein the median recess (3) is delimited by a bent or curved edge line and which is generally shaped in the form of an inverted “U”.

14. The concentration aid device (100) according to claim 1, wherein the lobes (41, 42) each have a width (l4) of between 10 and 20 centimeters.

15. Use of a concentration aid device (100) according to claim 1, for aiding the concentration of a person (12), wherein the headband (1) surrounds the forehead and the head (13) of this person (12), by positioning the median recess (3) in front of the eyes of the person (12) and by positioning the support strip (5) against the forehead to space the headband (1) away from the forehead of the person (12) thereby clearing a forward field of vision (10), which is laterally restricted by the two lobes (41, 42).

16. Use according to claim 15, wherein the median recess (3) is delimited by an upper edge (30), the eyes of the person (12) are aligned with a line of sight (120), and said headband (1) is positioned on the head (13) of the person (12) so that the upper edge (30) is positioned above the line of sight (120).

17. Use according to claim 16, wherein the upper edge (30) is positioned at a given distance (D2) above the line of sight (120), which distance (D2) is at most 3 centimeters.

18. The concentration aid device (100) according to claim 7, wherein the support strip (5) has a thickness (E5) of between 1.5 and 2.5 centimeters.

19. The concentration aid device (100) according to claim 14, wherein the lobes (41, 42) each have a width (l4) of between 13 and 18 centimeters.

20. Use according to claim 17, wherein the upper edge (30) is positioned at a given distance (D2) above the line of sight (120), which distance (D2) is between 1 and 2 centimeters.
